(19) United States
(12) Patent Application Publication
(10) Pub. No.: US 2010/0105090 A1
(43) Pub. Date: Apr. 29, 2010

(54) SECRETED LUCIFERASE MLUC7 AND USE THEREOF

(75) Inventors: Stefan Golz, Essen (DE); Eugene Vysotski, Krasnoyarsk (RU); Svetlana Markova, Krasnoyarsk (RU); Anna Tumenceva, Krasnoyarsk (RU)

Correspondence Address: ARENT FOX LLP, 1050 CONNECTICUT AVENUE, N.W., SUITE 400, WASHINGTON, DC, 20036, US

(73) Assignee: BAYER HEALTHCARE AG, Leverkusen (DE)

(21) Appl. No.: 12/526,016

(22) PCT Filed: Jan. 26, 2008
(86) PCT No.: PCT/EP08/00624

§ 371 Date: Nov. 30, 2009

(30) Foreign Application Priority Data
Feb. 6, 2007 (DE) 10 2007 005 803.0

(51) Int. Cl. C12Q 1/66; C07H 21/00; C12N 15/63; C12N 1/00; C12N 5/00; A01K 67/00; C12N 9/02; C07K 16/00

(52) U.S. Cl. 435/8; 536/23.2; 435/320.1; 435/243; 435/325; 800/13; 435/189; 530/387.9

(57) ABSTRACT

The invention relates to the nucleotide and amino acid sequences and to the activity and use of the secreted MLuc7 luciferase.

The invention relates to the nucleotide and amino acid sequences and to the activity and the use of the secreted MLuc7 luciferase and to the use of secreted luciferases.

### Luciferases

Luminescence refers to the emission of photons in the visible spectral range, which emission is due to excited emitter molecules. In contrast to fluorescence, the energy is not supplied externally here in the form of radiation of shorter wavelengths.

A distinction is made between chemiluminescence and bioluminescence. Chemiluminescence refers to a chemical reaction resulting in an excited molecule which itself luminesces when the excited electrons return to the ground state. If this reaction is catalysed by an enzyme, this is referred to as bioluminescence. The enzymes involved in the reaction are generally referred to as luciferases.

An overview of luminescent organisms can be found in Wilson & Hastings 1998.

Luciferases are peroxidases or mono- and dioxygenases. The enzyme substrates which are the starting substances for the light-emitting products are referred to as luciferins. They are different from species to species. The quantum yield of the systems is between 0.1-0.9 photons per substrate molecule converted.

Luciferases can be classified on the basis of their origin or their enzymic properties. Likewise, luciferases can be distinguished from one another by their substrate specificity. The most important substrates include coelenterazine and luciferin, and also derivatives of the two substances.

### Luciferase Substrates

The structures of some luciferase substrates are depicted below by way of example:

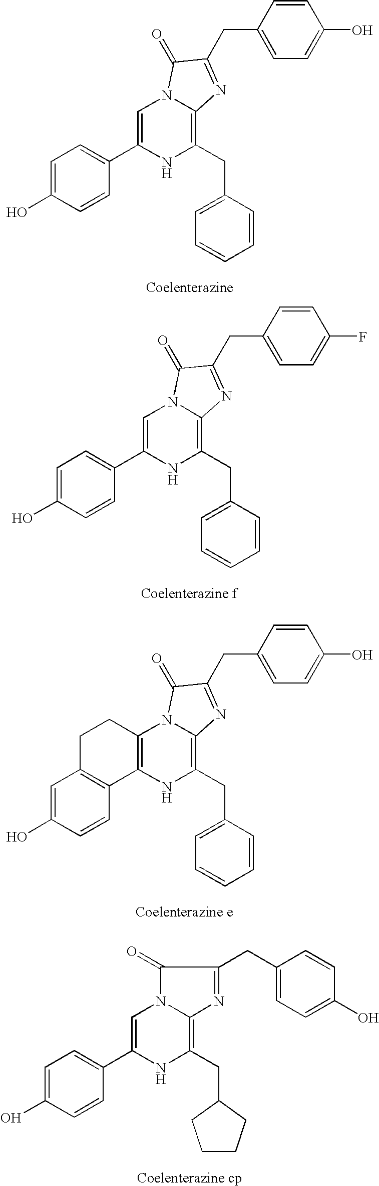

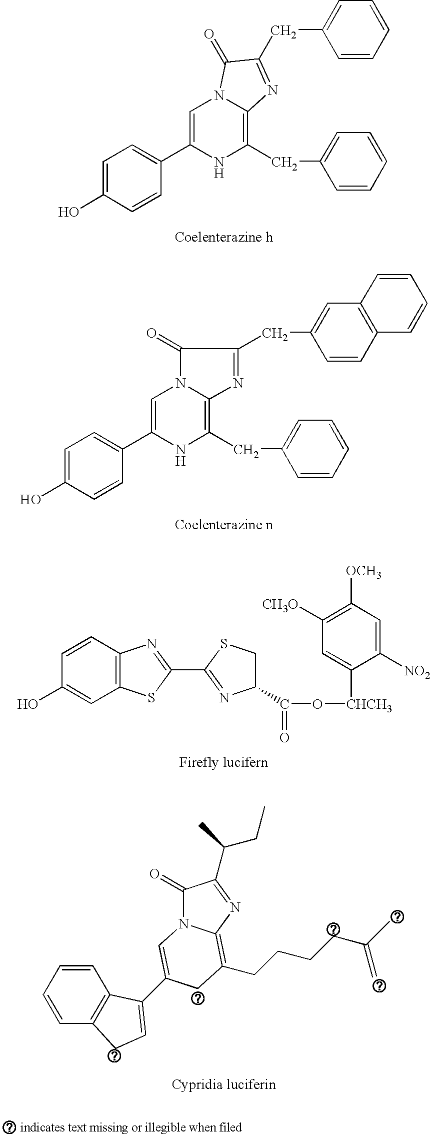

### Secreted Luciferases

Luciferases that are released in the form of a recombinant or wild-type protein by the host organism from the cytosol into the surrounding medium, are referred to as secreted luciferases. Table 1 gives an overview of secretory luciferases:

TABLE 1
Overview of secreted luciferases

| Luciferase | Organism | Reference |
|---|---|---|
| Lu164 | Metridia longa | WO0242470 A1 |
| Lu22 | Metridia longa | WO0242470 A1 |
| LuAL | Metridia longa | WO0242470 A1 |
| Lu39 | Metridia longa | WO0242470 A1 |
| Lu45 | Metridia longa | WO0242470 A1 |
| Lu16 | Metridia longa | WO0242470 A1 |
| Lu52 | Metridia longa | WO0242470 A1 |
| Cypridina luciferase | Cypridina Hilgendorfii | Tsuji et al. 1974 |
| Gaussia luciferase | Gaussia princeps | Christopoulos et al. 2002 |

The secreted Lu164 luciferase is likewise described in Markova et al. 2004.

### Reporter Systems

A reporter gene or indicator gene refers generally to genes whose gene products can be detected readily with the aid of simple biochemical or histochemical methods. At least 2 types of reporter genes are distinguished.

1. Resistance genes. Resistance genes refer to genes whose expression conveys to a cell resistance to antibiotics or other substances whose presence in the growth medium results in cell death, if the resistance gene is absent.
2. Reporter gene. The products of reporter genes are used in genetic engineering as fused or non-fused indicators. The most commonly used reporter genes include beta-galactosidase (Alam et al., 1990), alkaline phosphatase (Yang et al., 1997; Cullen et al., 1992), luciferases and other photoproteins (Shinomura, 1985; Phillips G N, 1997; Snowdowne et al., 1984).

Luminescence refers to the emission of photons in the visible spectral range, which emission is due to excited emitter molecules. In contrast to fluorescence, the energy is not supplied externally here in the form of radiation of shorter wavelengths.

A distinction is made between chemiluminescence and bioluminescence. Chemiluminescence refers to a chemical reaction resulting in an excited molecule which itself luminesces when the excited electrons return to the ground state. If this reaction is catalysed by an enzyme, this is referred to as bioluminescence. The enzymes involved in the reaction are generally referred to as luciferases.

### Secreted MLuc7 Luciferase

Surprisingly, when screening for new luciferases from Metridia longa, a new luciferase (referred to as MLuc7 hereinbelow) was identified and cloned whose biochemical and physicochemical properties clearly differ from the previously identified luciferases. These properties are described below:

### Kinetics

When expressing the secreted MLuc7 luciferase, the latter was surprisingly found to have a modified time resolution of the bioluminescence reaction (kinetics). The kinetic differences are substrate-independent for the substrates studied and depicted in FIGS. 8 and 9. FIG. 10 depicts the course of the bioluminescence reaction for MLuc7 and Lu164. The substantially faster kinetics of MLuc7 are clearly visible. MLuc7 exhibits a decrease of the luminescence to be measured per second, even after a few seconds, compared to Lu164. After 60 seconds, 70-80% of the integral signal of 300 seconds has already been recorded. Lu164 exhibits a substantially slower decrease in the bioluminescence signal per second, resulting in a distinct signal being measurable against the background even after 300 seconds. MLuc7 therefore differs kinetically from the previously described secreted luciferases of Metridia longa. Owing to this property, MLuc7 can surprisingly be utilised in combination with other coelenterazine-dependent or coelenterazine-independent luciferases, since kinetic distinction is possible.

### Activity

When expressing the secreted MLuc7 luciferase, the latter was surprisingly found to have an altered activity distribution of the bioluminescence reaction owing to the altered kinetic properties. At the start of the bioluminescence reaction, the MLuc7 activity to be measured per second is distinctly higher than that of Lu164. This higher bioluminescence makes possible higher sensitivity of the measurement method used, since a smaller number of cells, lower activation of MLuc7 expression or a lower substrate concentration makes possible a measurement distinctly above the background signal.

The invention relates to the use of MLuc7 for improving the sensitivity, the use of small cell numbers or low substrate concentrations.

### Kinetic Evaluation

The altered kinetic properties of MLuc7 make possible a differentiated kinetic evaluation of bioluminescence. With a continuous measurement over (for example) 300 seconds, various intervals can be used for evaluation. FIG. 13 depicts the bioluminescence signal totals for intervals of in each case 10 seconds. MLuc7 exhibits a distinctly higher bioluminescence than Lu164 within the first 60 seconds (the exact time period depends on the amount of luciferase and substrate used). After this period, the bioluminescence of MLuc7 decreases faster than that of Lu164, resulting in Lu164 having a higher bioluminescence signal. It is therefore possible to distinguish between the luciferases by way of choosing the measurement window. FIG. 14 depicts the bioluminescence totals of the MLuc7 and Lu164 luciferases for a time period of 300 seconds under the chosen experimental conditions.

FIG. 15 depicts the bioluminescence signal totals for intervals of in each case 60 seconds. Here too, the luciferases can be distinguished by way of choosing the measurement window. The length and selection of the measurement intervals can therefore adapt to the particular experimental conditions and be employed in a flexible manner. Owing to the data depicted, the total measurement time can also be chosen in a flexible manner.

The invention relates to the kinetic evaluation of measurements of the bioluminescence activity of MLuc7.

The invention relates to the kinetic evaluation of measurements of the bioluminescence activity of Lu164, Lu22, LuAL, Lu39, Lu45, Lu16 and Lu52.

The invention relates to the kinetic evaluation of measurements of the bioluminescence activity of secreted luciferases.

The invention relates to the kinetic evaluation of measurements of the bioluminescence activity of proteins according to the invention.

### Multiplexing

When expressing the secreted MLuc7 luciferase, the latter was surprisingly found to be particularly suitable for multiplex reactions, due to its altered properties. The MLuc7 luciferase exhibits distinctly faster kinetics in comparison with other luciferases, thereby making possible a combination with other luminescent or non-luminescent measurement methods (readouts).

In order to combine luminescent measurement methods, the luminescent systems must not inhibit each other or emit more light than the particular signals. After activating the first system (for example by adding substrate), luminescence must have returned to the starting level, before the second reaction can be started. This is also necessary if both systems use independent substrates. Due to its fast kinetics, MLuc7 shortens the time between the measurements markedly, Inactivation of the reaction is not necessary. Since the MLuc7 luciferase is a secreted luciferase, it may also be combined with intracellular systems (such as Firefly luciferase, for example).

Other Metridia longo luciferases can also be combined with intracellular systems such as Firefly luciferase. This however requires an inactivation step in order to lower the remaining bioluminescence to a low level.

The invention relates to the use of MLuc7 in multiplex reaction mixes in which a combination of MLuc7 with One or more reporter genes or measurement techniques (readouts) is used. The invention also relates to the use of Mluc7 in reaction mixes for measuring a plurality of target genes.

The invention relates to the use of Lu164, Lu22, LuAL, Lu39, Lu45, Lu16, and Lu52 in multiplex reaction mixes in which a combination of Lu164, Lu22, LuAL, Lu39, Lu45, Lu16 and Lu52 with one or more reporter genes or measurement techniques (readouts) is used. The invention also relates to the use of Lu164, Lu22, LuAL, Lu39, Lu45, Lu16 and Lu52 in reaction mixes for measuring a plurality of target genes.

The invention relates to the use of secreted luciferases in multiplex reaction mixes in which a combination of secreted luciferases with one or more reporter genes or measurement techniques (readouts) is used. The invention also relates to the use of secreted luciferases in reaction mixes for measuring a plurality of target genes.

The invention relates to the use of proteins according to the invention in multiplex reaction mixes in which a combination of proteins according to the invention with one or more reporter genes or measurement techniques (readouts) is used. The invention also relates to the use of proteins according to the invention in reaction mixes for measuring a plurality of target genes.

### Substrate Specificity

MLuc7 substrate specificity was studied by assaying various coelenterazines under standard conditions. This involved using supernatants from transient transfections of CHO cells of the Lu164, Lu22 and MLuc7 luciferases. The substrates coelenterazine n and cb will be converted more poorly by MLuc7 than by Lu164, and the substrate coelenterazine f will be converted better by Mluc than by Lu164, under the chosen conditions. The results demonstrate by way of example that the reaction can be optimised or the luciferases can be used on the basis of substrates and reaction conditions. The Firefly and Cypridina luciferin substrates are used as substrates by all three luciferases only to a small extent, if at all, under the chosen reaction conditions.

The invention relates to the use and combination of different substrates for generating bioluminescence by MLuc7.

The invention relates to the use and combination of different substrates for generating bioluminescence by Lu164, Lu22, LuAL, Lu39, Lu45, Lu16 and Lu52.

The invention relates to the use and combination of different substrates for generating bioluminescence by secreted luciferases.

The invention relates to the use and combination of different substrates for generating bioluminescence by proteins according to the invention.

### Temperature Dependence

The temperature dependence of the MLuc7 reaction was studied by measuring the bioluminescence reaction at temperatures of between 10 and 50° C. This involved using the supernatant from a transient transfection of CHO cells with MLuc7. The result indicates that the MLuc7 bioluminescence reaction is a function of the reaction temperature. This dependence can be used both for optimising and adapting the reaction in reporter gene applications and for distinguishing and combining various bioluminescent systems.

The invention relates to the use and combination of temperature dependence for developing and optimising measurement methods for MLuc7.

The invention relates to the use and combination of temperature dependence for developing and optimising measurement methods for Lu164, Lu22, LuAL, Lu39, Lu45, Lu16 and Lu52.

The invention relates to the use and combination of temperature dependence for developing and optimising measurement methods for secreted luciferases.

The invention relates to the use and combination of temperature dependence for developing and optimising measurement methods for proteins according to the invention.

### The Bioluminescence Reaction as a Function of Ion Concentration

The MLuc7 reaction as a function of ion concentration was studied by measuring the bioluminescence reaction with KCl concentrations of between 1 and 400 mM. This involved using the supernatant from a transient transfection of CHO cells with MLuc7. The result indicates that the MLuc7 bioluminescence reaction is a function of the ion concentration in the reaction medium. This dependence can be used both for optimising and adapting the reaction in reporter gene applications and for distinguishing and combining various bioluminescent systems.

The invention relates to the use and combination of ion dependence of the bioluminescence reaction for developing, optimising and using measurement methods for MLuc7.

The invention relates to the use and combination of ion dependence of the bioluminescence reaction for developing, optimising and using measurement methods for Lu164, Lu22, LuAL, Lu39, Lu45, Lu16 and Lu52.

The invention relates to the use and combination of ion dependence of the bioluminescence reaction for developing, optimising and using measurement methods for secreted luciferases.

The invention relates to the use and combination of ion dependence of the bioluminescence reaction for developing, optimising and using measurement methods for proteins according to the invention.

### Identification of the MLuc7 Luciferase

To study the bioluminescent activity of the species Metridia longa, specimens were caught in the White Sea (biological station Kartesh, Russia) and stored in liquid nitrogen. In order to prevent contaminations by other animal or plant species, 200 specimens of developmental stage V of Metridia longa were identified and stored as described above. Besides the “Naupilus” and the adult form, another five developmental forms of Metridia longa have been described, with forms of from CI to CV, at increasing developmental stage, being described by the nomenclature. Selection and identification were carried out with the aid of binocular microscopes and transfer pipettes. The specimens were caught in the White Sea in the region of the biological research station “Kartesh” (Russia).

Metridia longa individuals can be found at a depth that depends inter alia on their developmental state. This dependence is plotted in FIG. 19.

Besides fluctuations due to the seasons, salt content, temperature and food supply (composition and variety) and also other factors influence the habitat of Metridia longa. It is currently not known whether these factors influence metabolic processes or expression of bioluminescent proteins. A developmental stage-specific expression of bioluminescent proteins can also only be speculated about but is to be expected.

A specific study of individuals of selected developmental stages can therefore result in the identification of bioluminescent proteins which are expressed in other developmental stages to a distinctly lesser extent or not at all and which therefore are accessible to expression cloning only with limitations.

The invention relates to the study of bioluminescent organisms of specific developmental stages for identifying new bioluminescent proteins.

RNA was isolated from Metridia longa by using the Straight A's mRNA Isolation Kit (Novagen) according to the manufacturer's instructions. The isolated Poly-A mRNA was transcribed into cDNA with the aid of PowerScript Reverse Transcriptase (Clontech) and using the SMART cDNA Library Construction Kit (Clontech), according to the manufacturer's instructions. The expression vector used was the pTriplEx2 vector (Clontech), with the cDNA fragments being integrated into the SfiI A-B cleavage sites.

The expression vectors obtained were transformed with the aid of electroporation into E. coli XL1-Blue. The E. coli transformants were cultured under standard conditions.

The non-amplified cDNA library was plated with a colony density of about 1500 colonies per plate and incubated under standard conditions overnight. A copy of the bacteria plates was generated by applying a dry nitrocellulose membrane. The replicas were incubated under standard conditions. The colonies were picked from the replica plates with the aid of sterile glass rods and transferred to LB medium. The cultures were incubated to an optical density of 1 (at 600 nm) under standard conditions. This was followed by inducing gene expression by adding IPTG to a final concentration of 1 mM, followed by incubating at 37° C. for one hour. Three ml of the induced bacterial cultures were harvested by centrifugation, and the pellet was resuspended in 250 μl of SM buffer (100 mM NaCl, 10 mM MgCl2, 50 mM Tris-HCl pH 7.5, 0.01% gelatin). The bacteria were then disrupted by ultrasound treatment at 0° C. The crude extract was then studied.

To this end, coelenterazine (native) was added to a final concentration of 10 μM, and bioluminescence was determined in a luminometer. The cDNA of the bioluminescence-positive clones was sequenced with the aid of the ALFexpress II system according to the manufacturer's instructions (TermoSequenase Cy5 Dye Terminator Kit (GE Healthcare)).

Surprisingly, it was possible to identify with the aid of this method a Metridia longa luciferase of developmental stage V, referred to as MLuc7.

The invention relates to the secreted MLuc7 luciferase having the amino acid sequence represented by SEQ ID NO: 2. The invention likewise relates to the nucleic acid molecule depicted in SEQ ID NO: 1.

The invention also relates to functional equivalents of the secreted MLuc7 luciferase. Functional equivalents are proteins which have comparable physicochemical or biochemical properties.

The invention likewise relates to functional fragments of the MLuc7 protein and to nucleic acids coding for such fragments.

The invention likewise relates to mutants of the MLuc7 protein and to nucleic acids coding for such mutants.

The secreted MLuc7 luciferase is suitable as reporter gene for the “high content screening” (HCS) technique. HCS is a generic term for modern microscopy techniques for cell analysis. HCS processes are characterized by quantitatively recording a plurality of parameters at the cellular or subcellular level.

The secreted MLuc7 luciferase is suitable as reporter gene for cellular systems, especially for receptors, for ion channels, for transporters, for transcription factors or for inducible systems.

The secreted MLuc7 luciferase is suitable as reporter gene in bacterial and eukaryotic systems, especially in mammalian cells, in bacteria, in yeasts, in bakulo, in plants.

The secreted MLuc7 luciferase is suitable as reporter gene for cellular systems in combination with bioluminescent or chemoluminescent systems, especially systems with luciferases, with oxygenases, with phosphatases.

The secreted MLuc7 luciferase is suitable as reporter gene for cellular systems in combination with bioluminescent or chemoluminescent systems, especially systems with photoproteins and ion indicators, especially aequorin, clytin, obelin, berovin and bolinopsin.

The secreted MLuc7 luciferase is suitable as marker protein, especially in FACS (fluorescence-activated cell sorter) sorting.

The secreted MLuc7 luciferase is suitable as fusion proteins, especially for receptors, for ion channels, for transporters, for transcription factors, for proteinases, for kinases, for phosphodiesterases, for hydrolases, for peptidases, for transferases, for membrane proteins, for glycoproteins.

The secreted MLuc7 luciferase is suitable for immobilisation, especially by antibodies, by biotin, by magnetic or magnetisable supports.

The secreted MLuc7 luciferase is suitable for energy transfer systems, especially the FRET (fluorescence resonance energy transfer), BRET (bioluminescence resonance energy transfer), FET (field effect transistors), FP (fluorescence polarisation), HTRF (homogeneous time-resolved fluorescence) systems.

The secreted MLuc7 luciferase is suitable for labelling substrates or ligands, especially for proteases, for kinases, for transferases, for transporters, for ion channels and receptors.

The secreted MLuc7 luciferase is suitable for expression in bacterial systems, especially for determining titers, as substrates for biochemical systems, especially for proteinases and kinases.

The secreted MLuc7 luciferase is suitable as marker, especially coupled to antibodies, coupled to enzymes, coupled to receptors, coupled to ion channels and other proteins.

The secreted MLuc7 luciferase is suitable as reporter gene in pharmacological drug screening, especially in HTS (high throughput screening).

The secreted MLuc7 luciferase is suitable as components of detection systems, especially for ELISA (enzyme-linked immunosorbent assay), for immunohistochemistry, for Western blot, for confocal microscopy.

The secreted MLuc7 luciferase is suitable as marker for analysing interactions, especially for protein-protein interactions, for DNA-protein interactions, for DNA-RNA interactions, for RNA-RNA interactions, for RNA-protein interactions (DNA:deoxyribonucleic acid; RNA:ribonucleic acid).

The secreted MLuc7 luciferase is suitable as marker or fusion proteins for expression in transgenic organisms, especially in mice, in rats, in hamsters and other mammals, in primates, in fish, in worms, in plants.

The secreted MLuc7 luciferase is suitable as marker or fusion protein for analysing embryonic development.

The secreted MLuc7 luciferase is suitable as marker via a coupling mediator, especially via biotin, via NHS(N-hydroxysulphosuccinimide), via CN—Br.

The secreted MLuc7 luciferase is suitable as reporter coupled to nucleic acids, especially to DNA, to RNA.

The secreted MLuc7 luciferase is suitable as reporter coupled to proteins or peptides.

The nucleic acid or the peptide of the coupled MLuc7 protein is suitable as probe, especially for Northern blots, for Southern blots, for Western blots, for ELISA, for nucleic acid sequencing reactions, for protein analyses, for chip analyses.

The MLuc7 protein is suitable as label of pharmacological formulations, especially of infectious agents, of antibodies, of small molecules.

The MLuc7 protein is suitable for geological studies, especially for sea, groundwater and river currents.

The MLuc7 protein is suitable for expression in expression systems, especially in in-vitro translation systems, in bacterial systems, in yeast systems, in bakulo systems, in viral systems, in eukaryotic systems.

The invention also relates to purifying the MLuc7 protein, especially as wild-type protein, as fusion protein, as mutagenised protein.

The invention also relates to the use of MLuc7 in the field of cosmetics, especially of bath additives, of lotions, of soaps, of body paints, of toothpaste, of body powders.

The invention also relates to the use of Mluc7 for dyeing, especially of foodstuffs, of bath additives, of ink, of textiles, of plastics.

The invention also relates to the use of Mluc7 for dyeing of paper, especially of greetings cards, of paper products, of wallpapers, of handicraft articles.

The invention also relates to the use of Mluc7 for dyeing of liquids, especially for water pistols, for fountains, for beverages, for ice.

The invention also relates to the use of Mluc7 for the manufacture of toys, especially of fingerpaint, of make-up, water pistols.

The invention relates to organisms having a vector according to the invention.

The invention relates to organisms expressing a polypeptide according to the invention.

The invention relates to organisms expressing a functional equivalent of MLuc7.

The invention relates to methods of expressing the fluorescent polypeptides according to the invention in bacteria, eukaryotic cells or in in-vitro expression systems.

The invention also relates to methods of purifying/isolating a polypeptide according to the invention.

The invention relates to peptides having more than 5 consecutive amino acids which are recognised immunologically by antibodies to the fluorescent proteins according to the invention.

The invention relates to the use of the fluorescent proteins according to the invention as marker gene and reporter gene, in particular for pharmacological drug screening and diagnostics.

The invention relates to the secreted MLuc7 luciferase having the amino acid sequence represented by SEQ ID NO: 2 and the nucleotide sequence represented by SEQ ID NO: 1.

According to the invention, an MLuc7 protein is characterized in that its sequence comprises the sequence depicted in SEQ ID NO: 2 and functional fragments thereof.

The invention furthermore relates to a nucleic acid molecule which encodes a protein comprising the sequence depicted in SEQ ID NO: 1, and functional fragments thereof.

A recombinant RNA or DNA vector which comprises a nucleic acid as described in the previous paragraph is part of the invention.

A method of expressing a polypeptide according to the invention in bacteria, eukaryotic cells, or in in-vitro translation systems is part of the invention.

The use of a nucleic acid according to the invention as marker or reporter gene, also in combination with one or more other markers or reporter genes, is part of the invention.

The use of a protein according to the invention as marker or reporter gene, also in combination with one or more other markers or reporter gene proteins, is likewise part of the invention.

### Mutants and Derivatives of Secretory Luciferases

FIG. 3 depicts the alignment of the luciferases MLu7, the Metridia luciferases (Lu164, Lu22, LuAL, Lu39, Lu45, Lu16 and Lu52) and the Gaussia luciferase. The MLuc7 luciferase is a distinctly shorter polypeptide than the other luciferases analysed. The luciferases, Lu22 and Gaussia luciferase, likewise comprise distinctly shorter polypeptides.

The invention relates to mutants or derivatives of the luciferases Lu164, Lu22, LuAL, Lu39, Lu45, Lu16, Lu52 and Gaussia luciferase, having altered kinetic properties of the luminescence reaction.

The invention relates to mutants or derivatives, the modifications or deletions in the region of amino acids 23 to 78 of the luciferases Lu164, Lu22, LuAL, Lu39, Lu45, Lu16, Lu52 and Gaussia luciferase, having altered kinetic properties of the luminescence reaction.

The invention relates to mutants or derivatives, the modifications or deletions in the region of amino acids 23 to 78 of the luciferases Lu164, Lu22, LuAL, Lu39, Lu45, Lu16, Lu52 and Gaussia luciferase, having altered biochemical or physicochemical properties of the luminescence reaction.

The invention relates to mutants or derivatives, the modifications or deletions in the region of amino acids 13 to 88 of the luciferases Lu164, Lu22, LuAL, Lu39, Lu45, Lu16, Lu52 and Gaussia luciferase, having altered kinetic properties of the luminescence reaction.

The invention relates to mutants or derivatives, the modifications or deletions in the region of amino acids 13 to 88 of the luciferases Lu164, Lu22, LuAL, Lu39, Lu45, Lu16, Lu52 and Gaussia luciferase, having altered biochemical or physicochemical properties of the luminescence reaction.

The invention relates to mutants or derivatives, the modifications or deletions in the region of amino acids 33 to 68 of the luciferases Lu164, Lu22, LuAL, Lu39, Lu45, Lu16, Lu52 and Gaussia luciferase, having altered kinetic properties of the luminescence reaction.

The invention relates to mutants or derivatives, the modifications or deletions in the region of amino acids 33 to 68 of the luciferases Lu164, Lu22, LuAL, Lu39, Lu45, Lu16, Lu52 and Gaussia Luciferase, having altered biochemical or physicochemical properties of the luminescence reaction.

The invention relates in particular to:

1. A nucleic acid molecule selected from the group consisting of
a) nucleic acid molecules which encode a polypeptide comprising the amino acid sequence disclosed by SEQ ID NO: 2;
b) nucleic acid molecules which comprise the sequence depicted in SEQ ID NO: 1;
c) nucleic acid molecules whose complementary strand hybridises with a nucleic acid molecule of a) or b) under stringent conditions and which encode luciferases; a stringent hybridisation of nucleic acid molecules can be carried out for example in an aqueous solution which contains 0.2×SSC (1× standard saline-citrate=150 mM NaCl, 15 mM trisodium citrate) at 68° C. (Sambrook et al., 1989);
d) nucleic acid molecules which differ from those under c) due to the degeneracy of the genetic code;
e) nucleic acid molecules whose sequences are at least 70, 75, 80, 85, 95%, 98%, 99% identical to SEQ ID NO: 1 and whose protein products are luciferases;
f) nucleic acid molecules whose sequences are at least 65% identical to SEQ ID NO: 1 and which encode luciferases;
g) fragments of the nucleic acid molecules according to a)-f), which fragments encode functional luciferases.
2. A nucleic acid of point 1, which comprises a functional promoter 5′ of the photoprotein-encoding sequence.
3. Recombinant DNA or RNA vectors which comprise nucleic acids of point 2.
4. Organisms, comprising a vector according to point 3.
5. Oligonucleotides having more than 10 consecutive nucleotides which are identical or complementary to a subsequence of a nucleic acid molecule according to point 1,
6. Polypeptide encoded by a nucleic acid sequence of point 1.
7. Method of expressing the luciferase polypeptides according to point 6 in bacteria, eukaryotic cells or in in-vitro expression systems.
8. Method of purifying/isolating a luciferase polypeptide according to point 6.
9. Peptides having more than 5 consecutive amino acids which are recognised immunologically by antibodies to MLuc7 luciferase.
10. Use of a luciferase-encoding nucleic acid according to points 1 to 3 as marker gene or reporter gene.
11. Use of a luciferase according to point 6 as marker or reporter.
12. Antibody which specifically recognises a luciferase according to point 6.
13. Use according to point 10 or 11, wherein at least one further reporter gene is employed in addition to the MLuc7 luciferase.
14. Use according to point 13, wherein the further reporter gene(s) is(are) secreted and/or cellular luciferases.
15. Use according to point 14, wherein the further reporter gene(s) is(are) secreted luciferases.
16. Use according to point 14, wherein the further reporter gene(s) is(are) firefly luciferase or luciferases from the organism Metridia longa.
17. Use according to point 15, wherein the further secreted luciferases are luciferases selected from the group consisting of Lu164, Lu22, LuAL, Lu39, Lu45, Lu16 and Lu52.
18, Method according to point 10, 11, or 13, wherein the luminescence measurements are evaluated kinetically.
19. Method according to point 10, 11, 13 or 18, wherein a plurality of target proteins are measured.
20. A mutant or a derivative of a luciferase selected from the group consisting of the luciferases Lu164, Lu22, LuAL, Lu39, Lu45, Lu16, Lu52 and Gaussia luciferase, with altered kinetic properties of the luminescence reaction.
21, A mutant or a derivative of a luciferase selected from the group consisting of the luciferases Lu164, Lu22, LuAL, Lu39, Lu45, Lu16, Lu52 and Gaussia luciferase, which mutant or derivative has modifications or deletions in the region of amino acids 23 to 78 and altered kinetic properties of the luminescence reaction.
22. A mutant or a derivative of a luciferase selected from the group consisting of the luciferases Lu164, Lu22, LuAL, Lu39, Lu45, Lu16, Lu52 and Gaussia luciferase, which mutant or derivative has modifications or deletions in the region of amino acids 23 to 78 and altered biochemical or physicochemical properties of the luminescence reaction.
23. A mutant or a derivative of a luciferase selected from the group consisting of the luciferases Lu164, Lu22, LuAL, Lu39, Lu45, Lu16, Lu52 and Gaussia luciferase, which mutant or derivative has modifications or deletions in the region of amino acids 13 to 88 and altered kinetic properties of the luminescence reaction.
24. A mutant or a derivative of a luciferase selected from the group consisting of the luciferases Lu164, Lu22, LuAL, Lu39, Lu45, Lu16, Lu52 and Gaussia luciferase, which mutant or derivative has modifications or deletions in the region of amino acids 13 to 88 and altered biochemical or physicochemical properties of the luminescence reaction.
25. A mutant or a derivative of a luciferase selected from the group consisting of the luciferases Lu164, Lu22, LuAL, Lu39, Lu45, Lu16, Lu52 and Gaussia luciferase, which mutant or derivative has modifications or deletions in the region of amino acids 13 to 68 and altered kinetic properties of the luminescence reaction.
26. A mutant or a derivative of a luciferase selected from the group consisting of the luciferases Lu164, Lu22, LuAL, Lu39, Lu45, Lu16, Lu52 and Gaussia luciferase, which mutant or derivative has modifications or deletions in the region of amino acids 13 to 68 and altered biochemical or physicochemical properties of the luminescence reaction.

### Nucleotide and Amino Acid Sequences

(MLuc7-Nucleotide sequence-coding)

SEQ ID NO: 1

5′-ATGGATATCAAATTTATTTTTGCTCTTGTTTGCATTGCATTGGTCCA
GGCCAACCCTACTGTAAACAATGATGTTAACCGTGGTAAAATGCCTGGGA
AAAAATTGCCACTGGAAGTACTTATAGAAATGGAAGCCAATGCTTTTAAA
GCTGGCTGCACCAGGGGATGTCTCATTTGTCTTTCAAAAATCAAGTGCAC
AGCCAAAATGAAGCAGTACATTCCAGGAAGATGTCATGATTATGGAGGAG
ACAAGAAAACTGGACAGGCTGGAATAGTGGGTGCTATTGTTGACATTCCT
GAAATCTCTGGATTTAAGGAGATGGAACCAATGGAGCAGTTCATTGCTCA
AGTTGATCTCTGCGCCGACTGCACTACTGGCTGCCTCAAAGGTCTTGCCA
ATGTCAAGTGTTCTGAACTCCTCAAGAAATGGCTGCCAGACAGATGTGCA
AGTTTTGCTGACAAAATTCAAAAAGAAGCGCACAACATCAAGGGTCTTGC
TGGAGATCGT-3′

This results in an amino acid sequence of:

(MLuc7-Amino acid sequence)

SEQ ID NO: 2

MDIKFIFALVCIALVQANPTVNNDVNRGKMPGKKLPLEVLIEMEANAFKA
GCTRGCLICLSKIKCTAKMKQYIPGRCHDYGGDKKTGQAGIVGAIVDIPE
ISGFKEMEPMEQFIAQVDLCADCTTGCLKGLANVKCSELLKKWLPDRCAS
FADKIQKEAHNIKGLAGDR
(Lu164-Nucleotide sequence-coding)

SEQ ID NO: 3

5′-ATGGATATAAAGGTTGTCTTTACTCTTGTTTTCTCAGCATTGGTTCA
GGCAAAATCAACTGAATTCGATCCTAACATTGACATTGTTGGTTTAGAAG
GAAAATTTGGTATAACAAACCTTGAGACGGATTTATTCACAATATGGGAG
ACAATGGAGGTCATGATCAAAGCAGATATTGCAGATACTGATAGAGCCAG
CAACTTTGTTGCAACTGAAACCGATGCTAACCGTGGAAAAATGCCTGGCA
AAAAACTGCCACTGGCAGTTATCATGGAAATGGAAGCCAATGCTTTCAA
AGCTGGCTGCACCAGGGGATGCCTTATCTGTCTTTCAAAAAATAAAGTG
TACAGCCAAAATGAAGGTGTACATTCCAGGAAGATGTCATGATTATGGTG
GTGACAAGAAAACTGGACAGGCAGGAATAGTTGGTGCAATTGTTGACATTC
CCGAAATCTCTGGATTTAAGGAGATGGCACCCATGGAACAGTTCATTGCTC
AAGTTGAACGTTGCGCTTCCTGCACTACTGGATGTCTCAAAGGTCTTGCC
AATGTTAAGTGCTCTGAACTCCTGAAGAAATGGCTGCCTGACAGATGTGC
AAGTTTTGCTGACAAGATTCAAAAAGAAGTTCACAATATCAAAGGCATGG
CTGGAGATCGTTGA-3′

This results in an amino acid sequence of:

(LU164-Amino acid sequence)

SEQ ID NO: 4

MDIKVVFTLVFSALVQAKSTEFDPNIDIVGLEGKFGITNLETDLFTIWET
MEVMIKADIADTDRASNFVATBTDANRGKMPGKKLPLAVIMEMEANAFKA
GCTRGCLICLSKIKCTAKMKVYIPGRCHDYGGDKKTGQAGIVGAIVDIPE
ISGFKEMAPMEQFIAQVDRCASCTTGCLKGLANVKCSELLKKWLPDRCAS
FADKIQKEVHNIKGMAGDR
(MLuC7-Nucleotide sequence-cloned sequence)

SEQ ID NO: 5

5′-TGGTACCCGGGAATTCGGCCATTATGGCCGGGGATTCAGTCAACTGG
ATCCAAAAGGAAAGGTACTCCAAATATTGCCTGGAGGAAAAATGATATCAA
ATTTATTTTTGCTCTTGTTTGCATTGCATTGGTCCAGGCCAACCCTACTGT
AAACAATGATGTTAACCGTGGTAAAATGCCTGGGAAAAAATTGCCACTGGA
AGTACTTATAGAAATGGAGCCAATGCTTTTAAAGCTGGCTGCACCAGGGGC
ATGTCTCATTTGTCTTTCAAAAATCAAGTGCACAGCCAAAATGAAGCAGTA
CATTCCAGGAAGATGTCATGATTATGGAGGAGACAAGAAAACTGGACAGGC
TGGAATACTGGGTGCTATTGTTGACATTCCTGAAATCTCTGGATTTAAGGA
GATGGAACCAATGGAGCAGTTCATTGCTCAAGTTGTCTCTGCGCCGACTGC
ACTACTGGCTGCCTCAAAGGTCTTGCCAATGTCAAGTGTTCTGAACTCCTC
AAGAAATGGCTGCCAGACAGATGTGCAAGTTTTGCTGACAAAATTCAAAAA
GAAGCGCACAACATCAAGGGTCTTGCTGGAGATCGTTAAATAAACTGAGAA
AACAATGGATAACTGGATCAAGATAAGCTAATCTCATGATAAAAAATGGCC
AATTTAATTTAAAAATTATGAATTGTTAATTTTTATGTATGGAATTCCTTA
AATATATTCTATGTATTCAAAAAAAAAAAAAAAAAAAAAAAAAAAAACATG
TCGGCCGCCTCGGCCCAGTCGACTCTAGA-3′

### DESCRIPTION OF THE FIGURES

FIG. 1 depicts the vector map of the pcDNA3-MLuc7 construct.

FIG. 2 depicts vector map of the pASM-MLuc7 construct.

FIG. 3 depicts an alignment of various secreted luciferases at the amino acid level.

FIG. 4 depicts the substrate specificity of the Lu164, Lu22 and MLuc7 luciferases. X axis: coelenterazines, Y axis: relative light units (RLU). The activity was determined using in each case the same amounts of secreted luciferase from a transient transfection of CHO. The coelenterazines were added in parallel, and this was followed by starting the measurement.

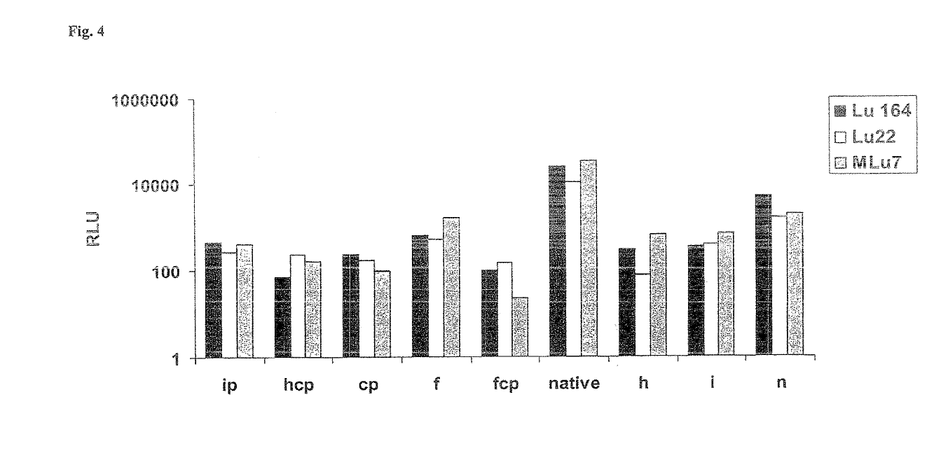

FIG. 5 depicts the temperature dependence of the MLuc7 luciferase. X axis: temperature in ° C., Y axis: relative light units (RLU). The activity was determined using in each case the same amounts of secreted luciferase from a transient transfection of CHO, The temperature indicated corresponds to the reaction temperature. The integral of the bioluminescence measurement of 60 seconds is plotted.

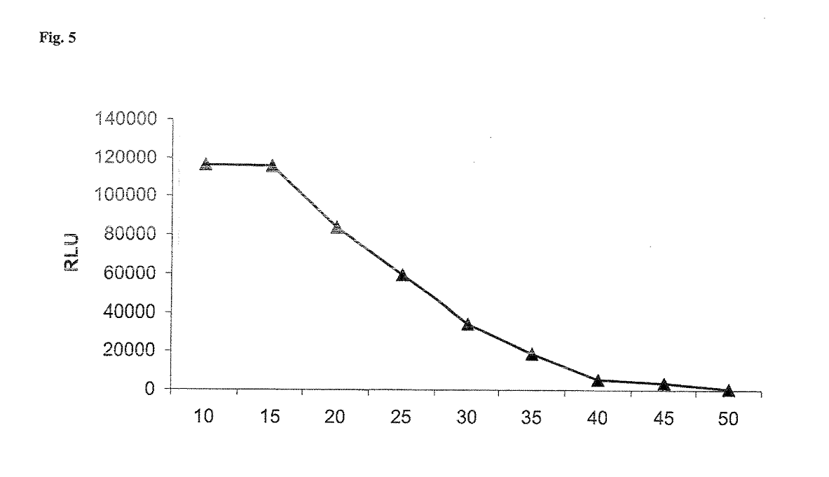

FIG. 6 depicts the dependence of the MLuc7 luciferase on the calcium chloride concentration in the reaction buffer. X axis: KCl concentration in mM, Y axis: relative light units (RLU). The activity was determined using in each case the same amounts of secreted luciferase from a transient transfection of CHO. The concentration indicated corresponds to the concentration of KCl in the reaction buffer. The integral of the bioluminescence measurement of 60 seconds is plotted.

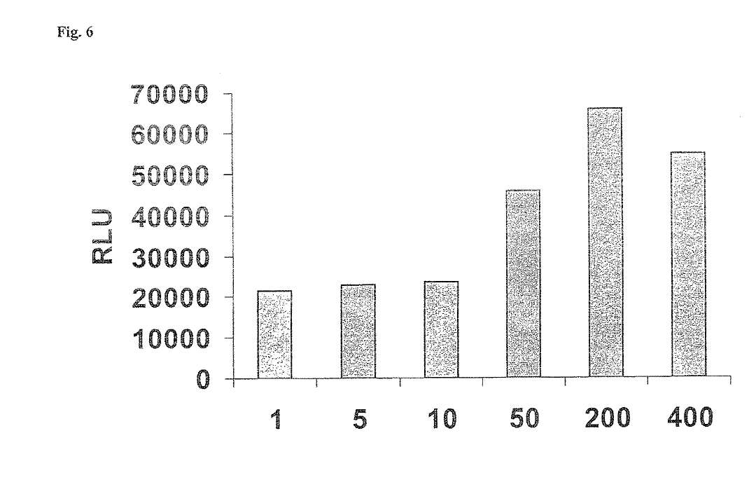

FIG. 7 depicts the bioluminescence measurement of MLuc7 with a constant amount of MLuc7 and different coelenterazine concentrations. X axis: coelenterazine concentration in μM. Y axis: relative light units (RLU).

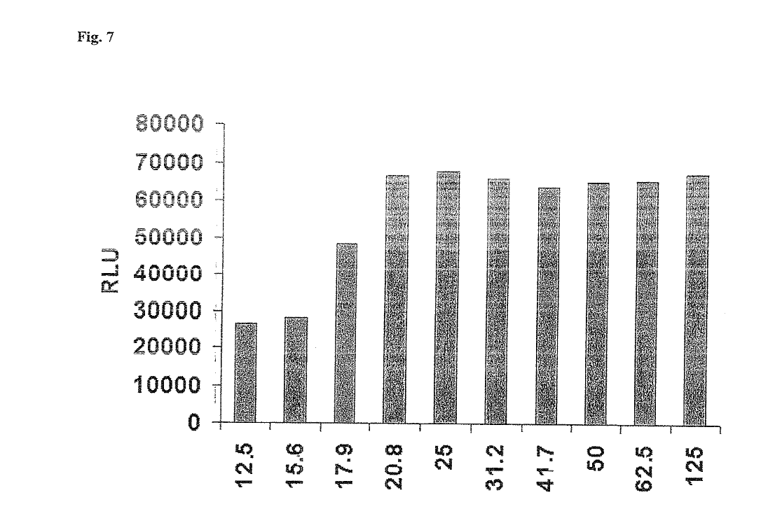

FIG. 8 The figure depicts the kinetics of the MLuc7 bioluminescence reaction with three different coelenterazines. X axis: time in seconds. Y axis: relative light units (RLU). Black: native coelenterazine, light grey: coelenterazine f, dark grey: coelenterazine i.

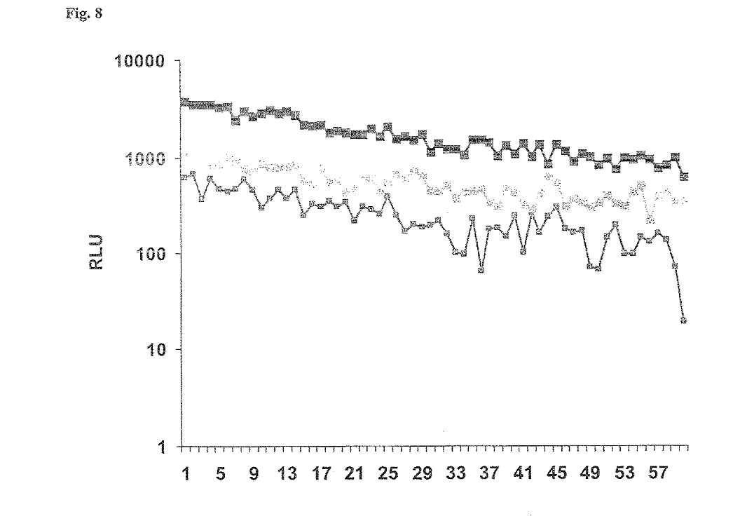

FIG. 9 The figure depicts the kinetics of the Lu164 bioluminescence reaction with three different coelenterazines. X axis: time in seconds. Y axis: relative light units (RLU). Black: native coelenterazine, light grey: coelenterazine f, dark grey: coelenterazine i.

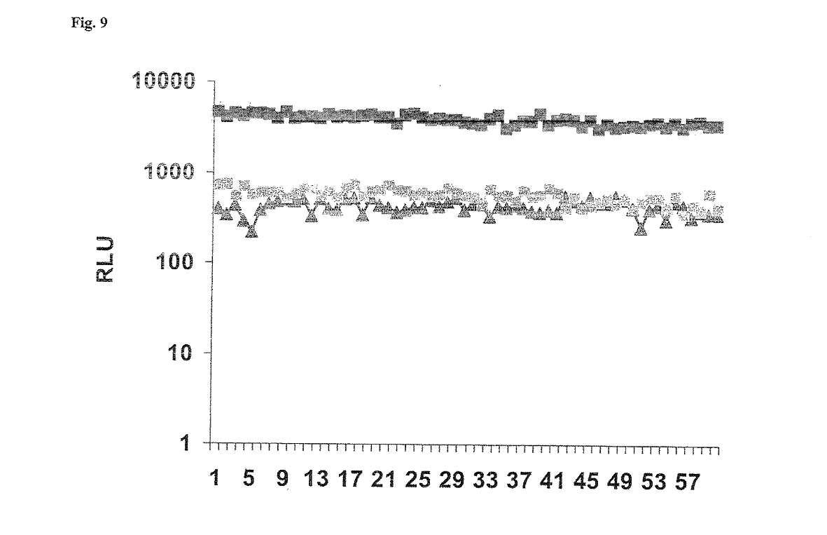

FIG. 10 depicts the result of the bioluminescence measurement of MLuc7 (black) and Lu164 (grey) for a measurement of 300 seconds with an integration time of 1.5 seconds. X axis: time in seconds, Y axis: relative light units (RLU).

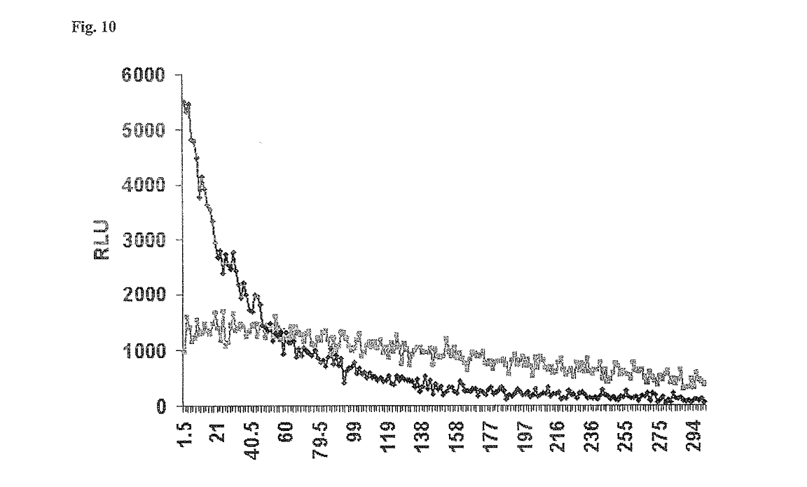

FIG. 11 depicts the result of the bioluminescence measurement of MLuc7 with a constant substrate concentration and decreasing Mluc7 concentration due to dilution of the cell supernatant. X axis; time in seconds. Y axis: relative light units (RLU). Box: indication and assignment of the dilution factor (starting from the undiluted supernatant).

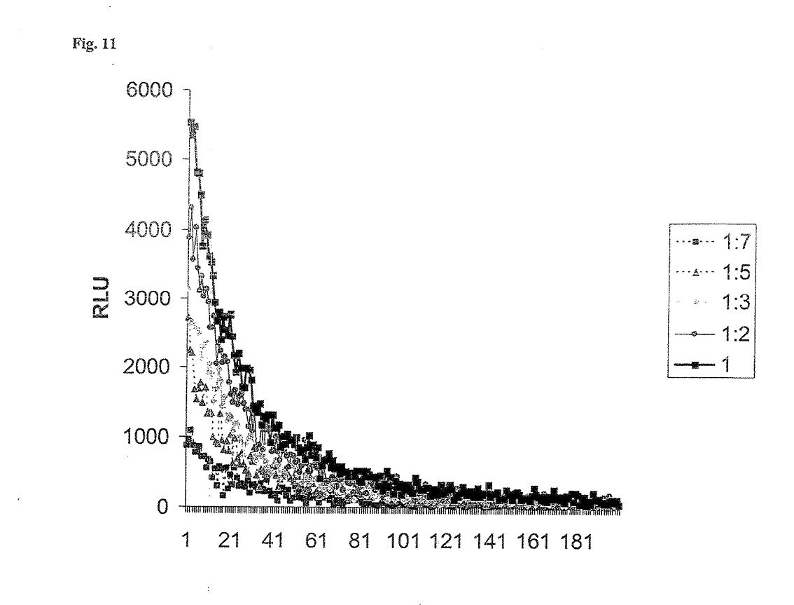

FIG. 12 depicts the result of the bioluminescence measurement of Lu164 with a constant substrate concentration and decreasing Lu164 concentration due to dilution of the cell supernatant. X axis: time in seconds. Y axis: relative light units (RLU). Box: indication and assignment of the dilution factor (starting from the undiluted supernatant).

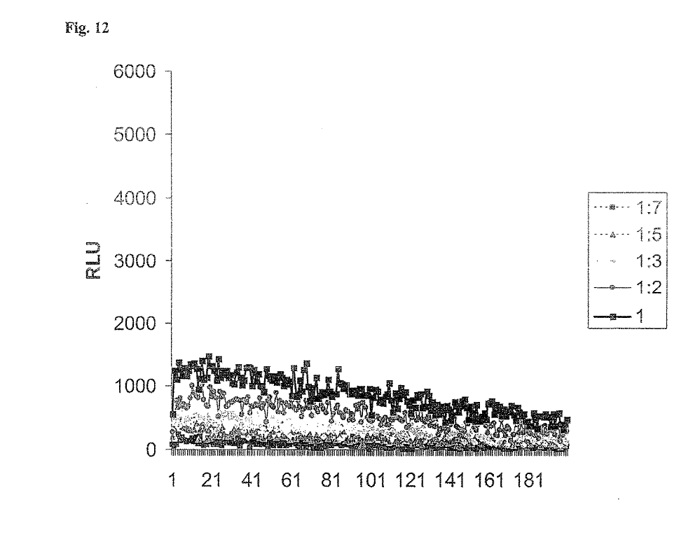

FIG. 13 depicts the result of the kinetic evaluation of Mluc7 and Lu164 bioluminescence measurements in segments of in each case 10 seconds of integration time. X axis: time in seconds. Y axis: relative light units (RLU).

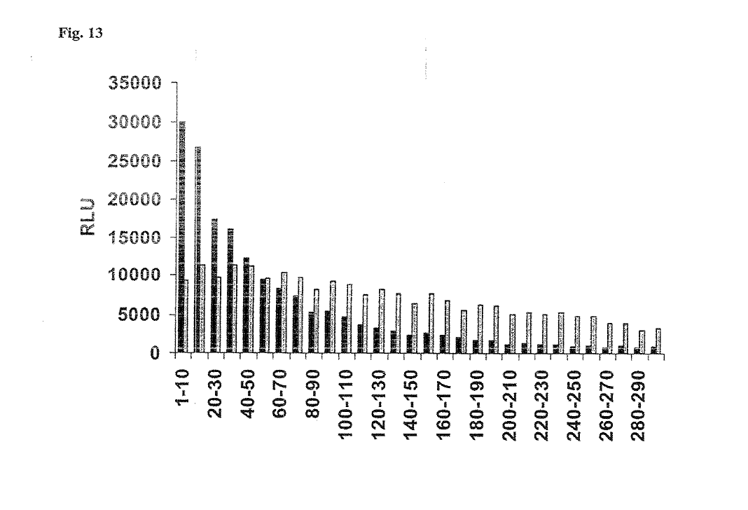

FIG. 14 depicts the result of the kinetic evaluation of MLuc7 and Lu164 bioluminescence measurements with an integration time of 300 seconds. X axis: time in seconds. Y axis: relative light units (RLU).

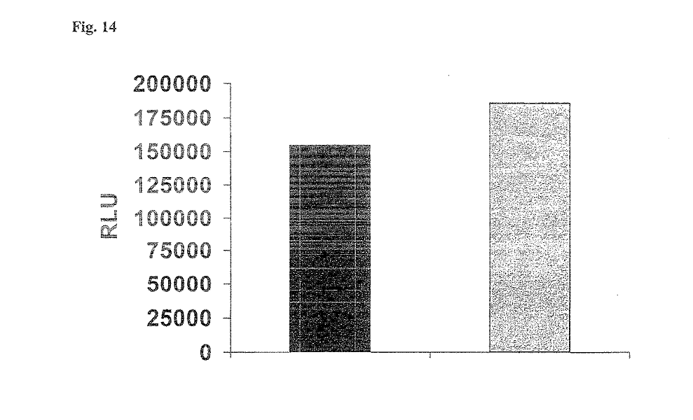

FIG. 15 depicts the result of the kinetic evaluation of Mluc7 and Lu164 bioluminescence measurements in segments of in each case 60 seconds of integration time. X axis: time in seconds. Y axis: relative light units (RLU).

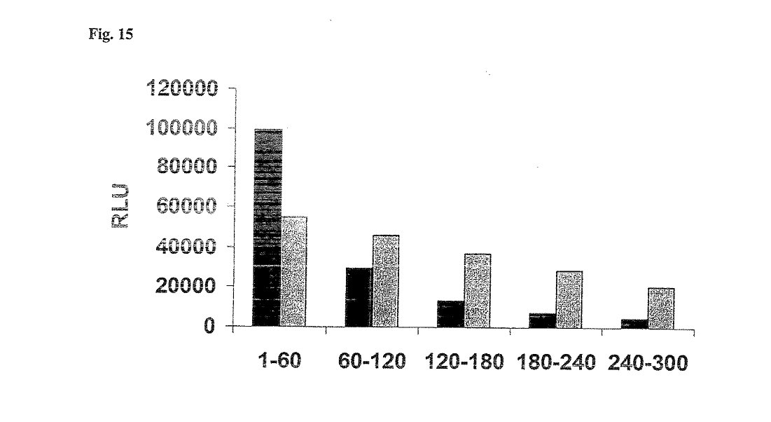

FIG. 16 depicts the water depth preferred by individuals of the species Metridia longa as a function of the developmental state. X axis: water depth in metres (m); Y axis: developmental state; black bars: 25-65 m, grey bars: 10-25 m, white bars: 0-10 m. Figure according to the information by National Oceanographic Data Center (USA).

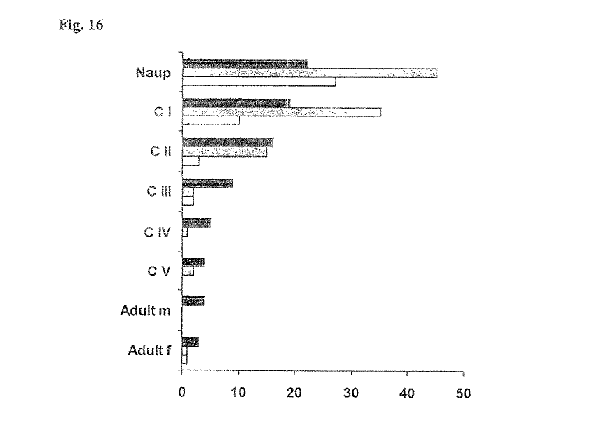

### EXAMPLE

### Example 1

### Preparation and Use of the Constructs

The vector used for preparing the construct described below was the pcDNA3.1(+) plasmid (Clontech) for constitutive expression. To detect changes in intracellular cAMP concentration, the MLac7 cDNA was cloned into the pASM vector. The pASM vector contains cAMP-responsive elements (CRE) which regulate promoter activity as a function of (AMP concentration. The derivative of said vector was referred to as pASM-MLuc7. The derivative of the pcDNA3 vector was referred to as pcDNA3-MLuc7. The cloning reactions were carried out using molecular-biological standard methods. The pcDNA3-Mluc7 and pASM-MLuc7 vectors were used for expressing MLuc7 in eukaryotic systems.

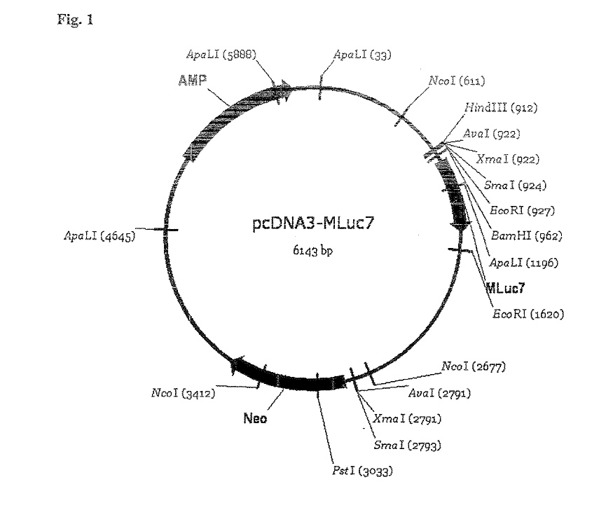

FIG. 1 depicts the plasmid map of the pcDNA3-Mluc7 vector.

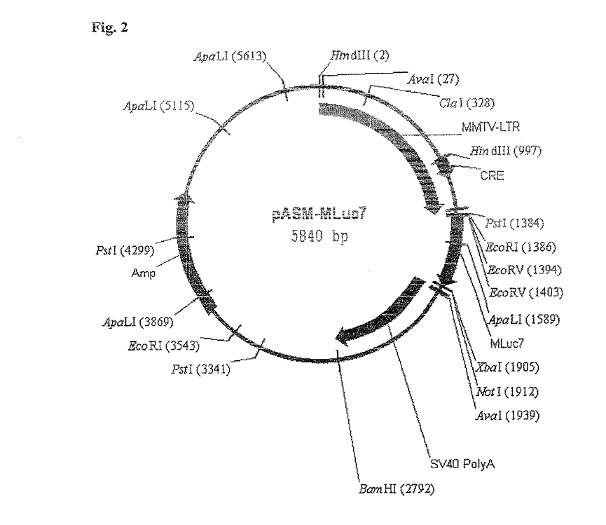

FIG. 2 depicts the plasmid map of the pASM-MLuc7 vector.

### Example 2

### Eukaryotic Expression

Constitutive eukaryotic expression was carried out in CHO cells by transfecting said cells with the expression plasmids pcDNA3-MLuc7, pcDNA3-Lu164 and pcDNA3 (without cDNA insert) in transient experiments. To this end, 10 000 cells per well in DMEM-F12 medium were plated on 96-well microtiter plates and incubated at 37° C. overnight. Transfection was carried out with the aid of the Fugene 6 kit (Roche) according to the manufacturer's instructions. The transfected cells were incubated in DMEM-F12 medium at 37° C. overnight. Bioluminescence was measured after the addition of substrate, using an imaging system. Diluted supernatants were measured in buffer A (pH 7.4) having the following composition: 130 mM NaCl, 5 mM KCl, 20 mM Hepes, 1 mM MgCl2×6H2O and 5 mM NaHCO3.

Stable cell lines were prepared by selecting the transfected cells with 2 mg/ml geneticin and determining the bioluminescence activity of the clones and supernatants, respectively.

### Example 3

### Sequence Comparison

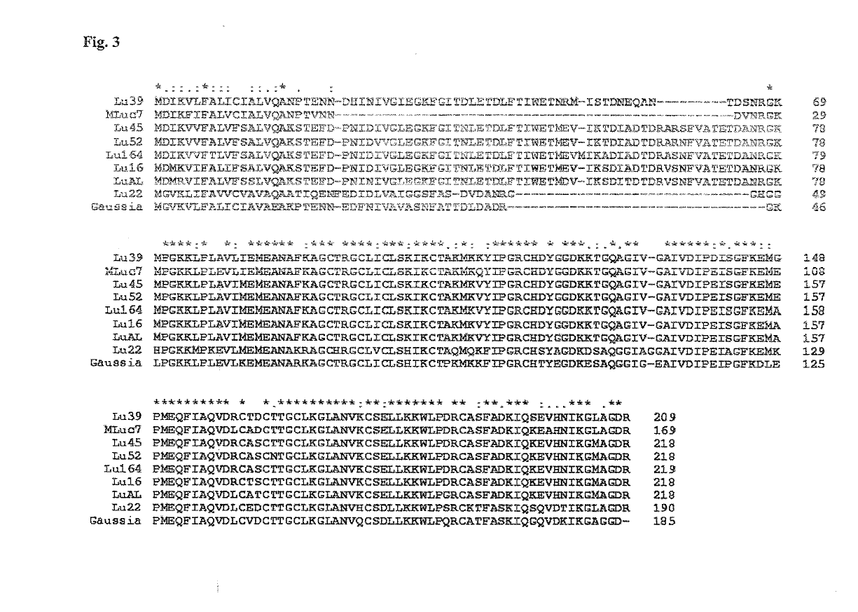

An amino acid sequence alignment was carried out in order to be able to compare and depict the sequences of the secreted luciferases. FIG. 3 depicts the alignment of the secreted luciferases at the amino acid level. Cypridina luciferase was not included in the alignment, since its sequence identity with the other luciferases is too low.

### LITERATURE/PATENTS

WO0242470 A1
Altschul, Stephen F., Thomas L. Madden, Alejandro A. Schäffer, Jinghui Zhang, Zheng Zhang, Webb Miller, and David J. Lipman (1997); Gapped BLAST and PSI-BLAST: a new generation of protein database search programs; Nucleic Acids Res. 25:3389-3402
Alam J, Cook J L. Reporter genes: application to the study of mammalian gene transcription. Anal Biochem. 1990 Aug. 1; 188(2):245-54
Christopoulos T K. Verhaegent M, Recombinant Gaussia luciferase. Overexpression, purification, and analytical application of a bioluminescent reporter for DNA hybridization. Anal Chem. 2002 Sep. 1; 74(17); 4378-85.
Cullen Bryan R., Malim Michael H., Secreted placental alkaline phosphatase as a eukaryotic reporter gene. Methods in Enzymology. 216:362ff
Svetlana V. Markova, Stefan Golz, Ludmila A. Frank, Bernd Kalthof, Eugene S. Vysotski, Cloning and Expression of cDNA for a Luciferase from the Marine Copepod Metridia longa, THE JOURNAL OF BIOLOGICAL CHEMISTRY Vol. 279, No. 5, Issue of January 30, pp. 3212-3217, 2004
Phillips G N. Structure and dynamics of green fluorescent protein. Curr Opin Struct Biol. 1997 December; 7(6):821-7
Shimomura O., Bioluminescence in the sea: photoprotein systems. Symp Soc Exp Biol. 1985; 39:351-72.
Snowdowne K W, Boric A B. Measurement of cytosolic free calcium in mammalian cells with aequorin. Am J Physiol. 1984 November; 247(5 Pt 1):C396-408.
Tsuji F I, Lynch R V 3rd, Stevens C L, Some properties of luciferase from the bioluminescent crustacean, Cypridina hilgendorfii. Biochemistry. 1974 Dec. 3; 13(25):5204-9.
Therese Wilson and J. Woodland Hastings, Bioluminescence, Annu. Rev. Cell Dev. Biol. 1998.14197-230
Yang Te-Tuan, Sinai Parisa, Kitts Paul A. Kain Seven R., Quantification of gene expression with a secreted alkaline phosphatase reporter system. Biotechnique. 1997 23(6) 1110ff

1. Nucleic acid molecule selected from the group consisting of
a) nucleic acid molecules which encode a polypeptide comprising the amino acid sequence disclosed by SEQ ID NO: 2;
b) nucleic acid molecules which comprise the sequence depicted in SEQ ID NO: 1;
c) nucleic acid molecules whose complementary strand hybridises with a nucleic acid molecule of a) or b) under stringent conditions and which encode luciferases;
d) nucleic acid molecules which differ from those under c) due to the degeneracy of the genetic code;
e) nucleic acid molecules whose sequences are at least 95% identical to SEQ ID NO: 1 and whose protein products are luciferases;
nucleic acid molecules whose sequences are at least 65% identical to SEQ ID NO: 1 and which encode luciferases;
g) fragments of the nucleic acid molecules according to a)-f), which fragments encode functional luciferases.

2. Nucleic acid of claim 1, which comprises a functional promoter 5′ of the photoprotein-encoding sequence.

3. Recombinant DNA or RNA vectors which comprise nucleic acids of claim 2.

4. Eukaryotic or prokaryotic cell or a non-human organism, comprising a vector according to claim 3.

5. Oligonucleotides having more than 10 consecutive nucleotides which are identical or complementary to a subsequence of a nucleic acid molecule according to claim 1.

6. Polypeptide encoded by a nucleic acid sequence of claim 1.

7. Method of expressing the luciferase polypeptides according to claim 6 in bacteria, eukaryotic cells or in in-vitro expression systems.

8. Method of purifying/isolating a luciferase polypeptide according to claim 6.

9. Peptides having more than 5 consecutive amino acids which are recognised immunologically by antibodies to MLuc7 luciferase.

10. Use of a luciferase-encoding nucleic acid according to claim 1 as marker gene or reporter gene.

11. Use of a luciferase according to claim 6 as marker or reporter.

12. Antibody which specifically recognises a luciferase according to claim 6.

13. Use according to claim 10, wherein at least one further reporter gene is employed in addition to the MLuc7 luciferase.

14. Use according to claim 13, wherein the further reporter gene(s) is(are) secreted and/or cellular luciferases.

15. Use according to claim 14, wherein the further reporter gene(s) is(are) secreted luciferases.

16. Use according to claim 14, wherein the further reporter gene(s) is(are) firefly luciferase or luciferases from the organism Metridia longa.

17. Use according to claim 15, wherein the further secreted luciferases are luciferases selected from the group consisting of Lu164, Lu22, LuAL, Lu39, Lu45, Lu16 and Lu52.

18. Method according to claim 10, wherein the luminescence measurements are evaluated kinetically.

19. Method according to claim 10, wherein a plurality of target proteins are measured.

20. A mutant or a derivative of a luciferase selected from the group consisting of the luciferases Lu164, Lu22, LuAL, Lu39, Lu45, Lu16, Lu52 and Gaussia luciferase, with altered kinetic properties of the luminescence reaction.

21. A mutant or a derivative of a luciferase selected from the group consisting of the luciferases Lu164, Lu22, LuAL, Lu39, Lu45, Lu16, Lu52 and Gaussia luciferase, which mutant or derivative has modifications or deletions in the region of amino acids 23 to 78 and altered kinetic properties of the luminescence reaction.

22. A mutant or a derivative of a luciferase selected from the group consisting of the luciferases Lu164, Lu22, LuAL, Lu39, Lu45, Lu16, Lu52 and Gaussia luciferase, which mutant or derivative has modifications or deletions in the region of amino acids 23 to 78 and altered biochemical or physicochemical properties of the luminescence reaction.

23. A mutant or a derivative of a luciferase selected from the group consisting of the luciferases Lu164, Lu22, LuAL, Lu39, Lu45, Lu16, Lu52 and Gaussia luciferase, which mutant or derivative has modifications or deletions in the region of amino acids 13 to 88 and altered kinetic properties of the luminescence reaction.

24. A mutant or a derivative of a luciferase selected from the group consisting of the luciferases Lu164, Lu22, LuAL, Lu39, Lu45, Lu16, Lu52 and Gaussia luciferase, which mutant or derivative has modifications or deletions in the region of amino acids 13 to 88 and altered biochemical or physicochemical properties of the luminescence reaction.

25. A mutant or a derivative of a luciferase selected from the group consisting of the luciferases Lu164, Lu22, LuAL, Lu39, Lu45, Lu16, Lu52 and Gaussia luciferase, which mutant or derivative has modifications or deletions in the region of amino acids 13 to 68 and altered kinetic properties of the luminescence reaction.

26. A mutant or a derivative of a luciferase selected from the group consisting of the luciferases Lu164, Lu22, LuAL, Lu39, Lu45, Lu16, Lu52 and Gaussia luciferase, which mutant or derivative has modifications or deletions in the region of amino acids 13 to 68 and altered biochemical or physicochemical properties of the luminescence reaction.


SEQUENCE LISTING (12 sequences)

SEQ ID NO: 1 - DNA, 507 bp, Metridia longa
atggatatcaaatttatttttgctcttgtttgcattgcattggtccaggccaaccctactgtaaacaatgatgttaaccgtggtaaaatgcctgggaaaaaattgccactggaagtacttatagaaatggaagccaatgcttttaaagctggctgcaccaggggatgtctcatttgtctttcaaaaatcaagtgcacagccaaaatgaagcagtacattccaggaagatgtcatgattatggaggagacaagaaaactggacaggctggaatagtgggtgctattgttgacattcctgaaatctctggatttaaggagatggaaccaatggagcagttcattgctcaagttgatctctgcgccgactgcactactggctgcctcaaaggtcttgccaatgtcaagtgttctgaactcctcaagaaatggctgccagacagatgtgcaagttttgctgacaaaattcaaaaagaagcgcacaacatcaagggtcttgctggagatcgt

SEQ ID NO: 2 - protein, 169 aa, Metridia longa
MDIKFIFALVCIALVQANPTVNNDVNRGKMPGKKLPLEVLIEMEANAFKAGCTRGCLICLSKIKCTAKMKQYIPGRCHDYGGDKKTGQAGIVGAIVDIPEISGFKEMEPMEQFIAQVDLCADCTTGCLKGLANVKCSELLKKWLPDRCASFADKIQKEAHNIKGLAGDR

SEQ ID NO: 3 - DNA, 660 bp, Metridia longa
atggatataaaggttgtctttactcttgttttctcagcattggttcaggcaaaatcaactgaattcgatcctaacattgacattgttggtttagaaggaaaatttggtataacaaaccttgagacggatttattcacaatatgggagacaatggaggtcatgatcaaagcagatattgcagatactgatagagccagcaactttgttgcaactgaaaccgatgctaaccgtggaaaaatgcctggcaaaaaactgccactggcagttatcatggaaatggaagccaatgctttcaaagctggctgcaccaggggatgccttatctgtctttcaaaaataaagtgtacagccaaaatgaaggtgtacattccaggaagatgtcatgattatggtggtgacaagaaaactggacaggcaggaatagttggtgcaattgttgacattcccgaaatctctggatttaaggagatggcacccatggaacagttcattgctcaagttgaacgttgcgcttcctgcactactggatgtctcaaaggtcttgccaatgttaagtgctctgaactcctgaagaaatggctgcctgacagatgtgcaagttttgctgacaagattcaaaaagaagttcacaatatcaaaggcatggctggagatcgttga

SEQ ID NO: 4 - protein, 219 aa, Metridia longa
MDIKVVFTLVFSALVQAKSTEFDPNIDIVGLEGKFGITNLETDLFTIWETMEVMIKADIADTDRASNFVATETDANRGKMPGKKLPLAVIMEMEANAFKAGCTRGCLICLSKIKCTAKMKVYIPGRCHDYGGDKKTGQAGIVGAIVDIPEISGFKEMAPMEQFIAQVDRCASCTTGCLKGLANVKCSELLKKWLPDRCASFADKIQKEVHNIKGMAGDR

SEQ ID NO: 5 - DNA, 789 bp, Metridia longa
tggtacccgggaattcggccattatggccggggattcagtcaactggatccaaaaggaaaggtactccaaatattgcctggaggaaaaatgatatcaaatttatttttgctcttgtttgcattgcattggtccaggccaaccctactgtaaacaatgatgttaaccgtggtaaaatgcctgggaaaaaattgccactggaagtacttatagaaatggagccaatgcttttaaagctggctgcaccaggggatgtctcatttgtctttcaaaaatcaagtgcacagccaaaatgaagcagtacattccaggaagatgtcatgattatggaggagacaagaaaactggacaggctggaatagtgggtgctattgttgacattcctgaaatctctggatttaaggagatggaaccaatggagcagttcattgctcaagttgtctctgcgccgactgcactactggctgcctcaaaggtcttgccaatgtcaagtgttctgaactcctcaagaaatggctgccagacagatgtgcaagttttgctgacaaaattcaaaaagaagcgcacaacatcaagggtcttgctggagatcgttaaataaactgagaaaacaatggataactggatcaagataagctaatctcatgataaaaaatggccaatttaatttaaaaattatgaattgttaatttttatgtatggaattccttaaatatattctatgtattcaaaaaaaaaaaaaaaaaaaaaaaaaaaaacatgtcggccgcctcggcccagtcgactctaga

SEQ ID NO: 6 - protein, 209 aa, Metridia longa
MDIKVLFALICIALVQANPTENNDHINIVGIEGKFGITDLETDLFTIWETNRMISTDNEQANTDSNRGKMPGKKLPLAVLIEMEANAFKAGCTRGCLICLSKIKCTAKMKKYIPGRCHDYGGDKKTGQAGIVGAIVDIPDISGFKEMGPMEQFIAQVDRCTDCTTGCLKGLANVKCSELLKKWLPDRCASFADKIQSEVHNIKGLAGDR

SEQ ID NO: 7 - protein, 218 aa, Metridia longa
MDIKVVFALVFSALVQAKSTEFDPNIDIVGLEGKFGITNLETDLFTIWETMEVIKTDIADTDRARSFVATETDANRGKMPGKKLPLAVIMEMEANAFKAGCTRGCLICLSKIKCTAKMKVYIPGRCHDYGGDKKTGQAGIVGAIVDIPEISGFKEMEPMEQFIAQVDRCASCTTGCLKGLANVKCSELLKKWLPDRCASFADKIQKEVHNIKGMAGDR

SEQ ID NO: 8 - protein, 218 aa, Metridia longa
MDIKVVFALVFSALVQAKSTEFDPNIDVVGLEGKFGITNLETDLFTIWETMEVIKTDIADTDRARNFVATETDANRGKMPGKKLPLAVIMEMEANAFKAGCTRGCLICLSKIKCTAKMKVYIPGRCHDYGGDKKTGQAGIVGAIVDIPEISGFKEMEPMEQFIAQVDRCASCNTGCLKGLANVKCSELLKKWLPDRCASFADKIQKEVHNIKGMAGDR

SEQ ID NO: 9 - protein, 218 aa, Metridia longa
MDMKVIFALIFSALVQAKSTEFDPNIDIVGLEGKFGITNLETDLFTIWETMEVIKSDIADTDRVSNFVATETDANRGKMPGKKLPLAVIMEMEANAFKAGCTRGCLICLSKIKCTAKMKVYIPGRCHDYGGDKKTGQAGIVGAIVDIPEISGFKEMAPMEQFIAQVDRCTSCTTGCLKGLANVKCSELLKKWLPDRCASFADKIQKEVHNIKGMAGDR

SEQ ID NO: 10 - protein, 218 aa, Metridia longa
MDMRVIFALVFSSLVQAKSTEFDPNINIVGLEGKFGITNLETDLFTIWETMDVIKSDITDTDRVSNFVATETDANRGKMPGKKLPLAVIMEMEANAFKAGCTRGCLICLSKIKCTAKMKVYIPGRCHDYGGDKKTGQAGIVGAIVDIPEISGFKEMAPMEQFIAQVDLCATCTTGCLKGLANVKCSELLKKWLPGRCASFADKIQKEVHNIKGMAGDR

SEQ ID NO: 11 - protein, 190 aa, Metridia longa
MGVKLIFAVVCVAVAQAATIQENFEDIDLVAIGGSFASDVDANRGGHGGHPGKKMPKEVLMEMEANAKRAGCHRGCLVCLSHIKCTAQMQKFIPGRCHSYAGDKDSAQGGIAGGAIVDIPEIAGFKEMKPMEQFIAQVDLCEDCTTGCLKGLANVHCSDLLKKWLPSRCKTFASKIQSQVDTIKGLAGDR

SEQ ID NO: 12 - protein, 185 aa, Gaussia princeps
MGVKVLFALICIAVAEAKPTENNEDFNIVAVASNFATTDLDADRGKLPGKKLPLEVLKEMEANARKAGCTRGCLICLSHIKCTPKMKKFIPGRCHTYEGDKESAQGGIGEAIVDIPEIPGFKDLEPMEQFIAQVDLCVDCTTGCLKGLANVQCSDLLKKWLPQRCATFASKIQGQVDKIKGAGGD